(19) United States
(12) Patent Application Publication
(10) Pub. No.: US 2025/0195823 A1
(43) Pub. Date: Jun. 19, 2025

(54) EMDR Therapy Tactile Stimulation Device

(71) Applicant: Alyson Bye, Odessa, TX (US)

(72) Inventor: Alyson Bye, Odessa, TX (US)

(21) Appl. No.: 18/432,278

(22) Filed: Feb. 5, 2024

Related U.S. Application Data
(60) Provisional application No. 63/611,252, filed on Dec. 18, 2023.

(51) Int. Cl. A61M 21/02; A61H 23/02; A61M 21/00

(52) U.S. Cl. CPC A61M 21/02; A61H 23/02; A61H 2201/0153; A61H 2201/169; A61H 2201/5005; A61H 2201/5025; A61H 2201/5043; A61H 2205/065; A61M 2021/0022

(57) ABSTRACT

An EMDR therapy tactile stimulation device is provided. The device is comprised of a handheld buzzer with at least one vibration motor. The vibration motor can be remotely controlled via a mobile application by a therapist during a remote EMDR therapy session. As a result, the therapist can remotely deliver tactile simulation via vibration to at least one hand of the user as needed.

### CROSS-REFERENCE TO RELATED APPLICATION

The present application claims priority to, and the benefit of, U.S. Provisional Application No. 63/611,252, which was filed on Dec. 18, 2023, and is incorporated herein by reference in its entirety.

### FIELD OF THE INVENTION

The present invention relates generally to the field of therapy devices. More specifically, the present invention relates to an EMDR therapy tactile stimulation device that can be held in a user's hand and remotely delivers stimulation during EMDR therapy. Accordingly, the present disclosure makes specific reference thereto. Nonetheless, it is to be appreciated that aspects of the present invention are also equally applicable to other like applications, devices, and methods of manufacture.

### BACKGROUND

Eye Movement Desensitization and Reprocessing (EMDR) is a distinctive form of psychotherapy developed to help individuals recover from the impact of traumatic experiences. Central to this approach is the use of bilateral stimulation, traditionally involving guided eye movements. However, EMDR therapy has evolved to include other forms of bilateral stimulation, such as auditory tones or tactile vibrations. This method employs gentle, rhythmic tactile stimuli that is often delivered through handheld devices that a client holds in each hand. These vibrations alternate between the left and right hand, mirroring the back-and-forth eye movements originally used in EMDR.

The use of vibration offers several advantages, especially in virtual therapy settings where traditional eye movement techniques might be challenging to implement effectively due to the limitations of digital communication. Vibration can be a more feasible option for remote therapy, as it does not rely on the visual connection between therapist and client. This method maintains the core principle of bilateral stimulation, essential for the EMDR process, which is thought to facilitate the brain's information processing system, aiding in the integration of traumatic memories. However, clients may not have access to the necessary tactile stimulation equipment and understand how to use it properly during EMDR therapy.

Therefore, there exists a long-felt need in the art for a device that can be used during EMDR therapy. There also exists a long-felt need in the art for an EMDR therapy tactile stimulation device. More specifically, there exists a long-felt need in the art for an EMDR therapy tactile stimulation device that can be used during EMDR therapy and wherein the device provides bilateral stimulation.

The subject matter disclosed and claimed herein, in one embodiment thereof, comprises an EMDR therapy tactile stimulation device. The device is comprised of a handheld buzzer with at least one vibration motor. The vibration motor can be remotely controlled via a mobile application by a therapist during a remote EMDR therapy session. As a result, the therapist can remotely deliver tactile simulation via vibration to at least one hand of the user as needed.

In this manner, the EMDR therapy tactile stimulation device of the present invention accomplishes all the foregoing objectives and provides a device that can be used during EMDR therapy. More specifically, the device can be used during EMDR therapy wherein the device provides bilateral stimulation in the form of vibration.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an EMDR therapy tactile stimulation device. The device is comprised of at least one buzzer comprised of at least one vibration motor that can be remotely controlled via at least one mobile application.

The buzzer is comprised of a body comprised of at least one contoured area that receives the hand/fingers of a user. The body is comprised of at least one motor that produces at least one vibration. The motor is powered by at least one battery.

The buzzer is comprised of at least one button that powers the motor on. Once powered on, at least one indicator light may illuminate to indicate that the buzzer is ready for use. The buzzer is also comprised of at least one receiver. The device is further comprised of at least one mobile application that can be installed on a smart device such as, but not limited to, a smartphone, a computer, a tablet, etc., wherein the smart device is in wireless electrical communication (i.e., Wifi, Bluetooth, etc.) with the receiver. The mobile application can be used by a therapist to remotely control the vibration of the motor.

More specifically, the application allows a user to control the frequency of vibration of the motor. The application also allows a user to control the intensity of the vibration of the motor. In an embodiment wherein the device is comprised of two buzzers that are in wired or wireless communication, the application may allow a user to select which buzzer receives the vibration from the motor. As a result, the therapist can remotely and virtually use the application and buzzer to bilaterally stimulate a user during an EMDR therapy session.

The present invention is also comprised of a method of using the device. First, a device is provided comprised of at least one buzzer comprised of at least one motor having at least one battery, at least one receiver, and a mobile application. Then, the receiver can be paired with the mobile application via wireless electrical communication. Next, a first user can hold the buzzer in their hand. Finally, a second user (such as, but not limited to, a therapist) can use the mobile application to configure the motor to deliver at least one vibration to the first user via the buzzer during a remote, virtual EMDR therapy session.

Accordingly, the EMDR therapy tactile stimulation device of the present invention is particularly advantageous as it provides a device that can be used during EMDR therapy. More specifically, the device can be used during EMDR therapy wherein the device provides bilateral stimulation in the form of vibration. In this manner, the EMDR therapy tactile stimulation device overcomes the limitations of existing methods of virtual EMDR therapy known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front and top view of a buzzer of one potential embodiment of an EMDR therapy tactile stimulation device of the present invention in accordance with the disclosed architecture;

FIG. 2 illustrates a front view of two connected buzzers of one potential embodiment of an EMDR therapy tactile stimulation device of the present invention in accordance with the disclosed architecture;

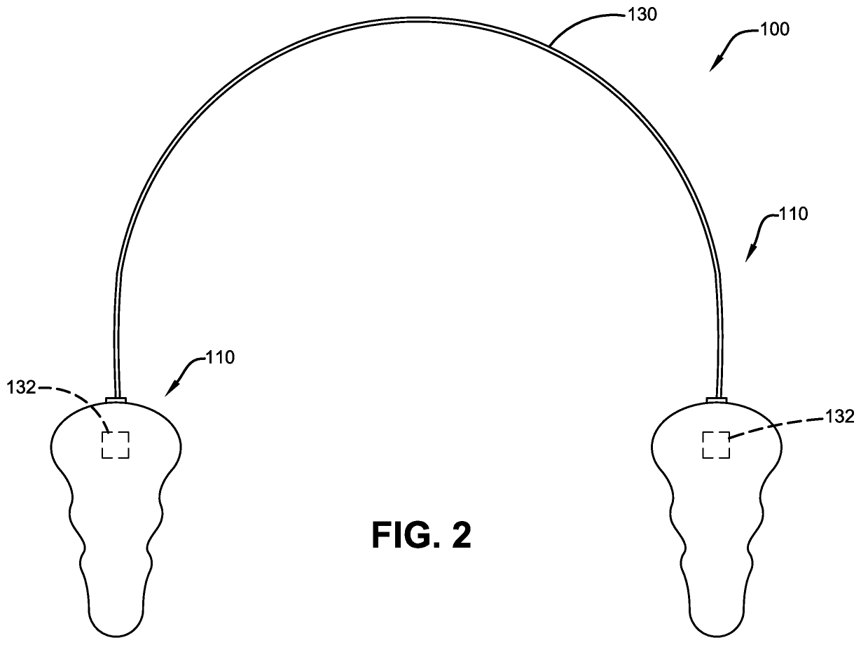

FIG. 3 illustrates a graphical view of a mobile application of one potential embodiment of an EMDR therapy tactile stimulation device of the present invention in accordance with the disclosed architecture; and

FIG. 4 illustrates a flowchart of a method of using one potential embodiment of an EMDR therapy tactile stimulation device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that can be used during EMDR therapy. There also exists a long-felt need in the art for an EMDR therapy tactile stimulation device. More specifically, there exists a long-felt need in the art for an EMDR therapy tactile stimulation device that can be used during EMDR therapy and wherein the device provides bilateral stimulation.

The present invention, in one exemplary embodiment, is comprised of an EMDR therapy tactile stimulation device comprised of at least one buzzer comprised of at least one vibration motor that can be remotely controlled via at least one mobile application. The buzzer is comprised of a body comprised of at least one contoured area that receives the hand/fingers of a user. The body is comprised of at least one motor that produces at least one vibration and that is powered by at least one battery.

The buzzer is comprised of at least one button that powers the motor on. Once powered on, at least one indicator light may illuminate to indicate that the buzzer is ready for use. The buzzer is also comprised of at least one receiver, wherein the device is further comprised of at least one mobile application that can be installed on a smart device such as, but not limited to, a smartphone, a computer, a tablet, etc. The smart device can then be placed in wireless electrical communication (i.e., Wifi, Bluetooth, etc.) with the receiver. The mobile application can be used by a therapist to remotely control the vibration of the motor.

More specifically, the application allows a user to control the frequency of vibration of the motor. The application also allows a user to control the intensity of the vibration of the motor. In an embodiment wherein the device is comprised of two buzzers that are in wired or wireless communication, the application may allow a user to select which buzzer receives the vibration from the motor. As a result, the therapist can remotely and virtually use the application and buzzer to bilaterally stimulate a user during an EMDR therapy session.

The present invention is also comprised of a method of using the device. First, a device is provided comprised of at least one buzzer comprised of at least one motor having at least one battery, at least one receiver, and a mobile application. Then, the receiver can be paired with the mobile application via wireless electrical communication. Next, a first user can hold the buzzer in their hand. Finally, a second user (such as, but not limited to, a therapist) can use the mobile application to configure the motor to deliver at least one vibration to the first user via the buzzer during a remote, virtual EMDR therapy session.

Accordingly, the EMDR therapy tactile stimulation device of the present invention is particularly advantageous as it provides a device that can be used during EMDR therapy. More specifically, the device can be used during EMDR therapy wherein the device provides bilateral stimulation in the form of vibration. In this manner, the EMDR therapy tactile stimulation device overcomes the limitations of existing methods of virtual EMDR therapy known in the art.

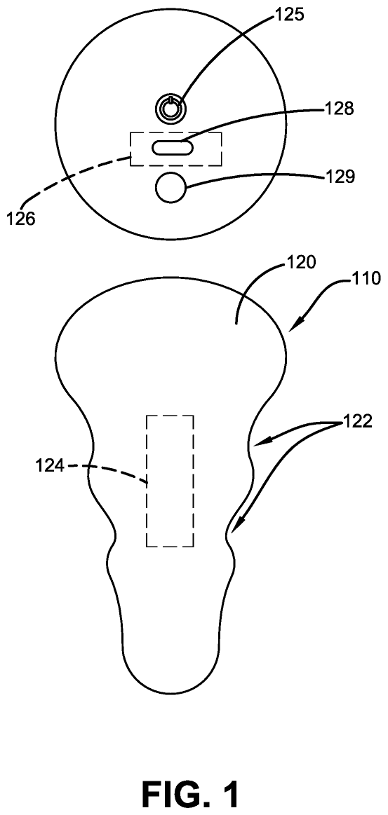

Referring initially to the drawings, FIG. 1 illustrates a front and top view of a buzzer of one potential embodiment of an EMDR therapy tactile stimulation device 100 of the present invention in accordance with the disclosed architecture. The device 100 is comprised of at least one buzzer 110 comprised of at least one vibration motor 124 that can be remotely controlled via at least one mobile application 140.

The buzzer 110 is comprised of a body 120 that may be any shape, size, and configuration in different embodiments. In one embodiment, the body 120 may be comprised of at least one contoured area 122 that receives the hand/fingers of a user. The body 120 may be made from any material such as, but not limited to, rubber, plastic, etc.

The body 120 is comprised of at least one motor 124. The motor 124 produces at least one vibration. The motor 124 is powered by at least one battery 126. The battery 126 may be removable or non-removable from the body 110 in different embodiments. The battery 126 may also be disposable or rechargeable. In a non-removable and rechargeable embodiment, the battery 126 can be recharged via at least one charging port 128, such as, but not limited to, a USB port.

The buzzer 110 is comprised of at least one button 125 that powers on the motor 124. Once powered on, at least one indicator light 129 may illuminate to indicate that the buzzer 110 is on. The indicator light 129 may also illuminate to communicate the battery 126 level. The light 129 may illuminate any color(s).

The buzzer 110 is comprised of at least one receiver 132. The device 100 is also comprised of at least one mobile application 140. The application 140 can be installed on a smart device such as, but not limited to, a smartphone, a computer, a tablet, etc., wherein the smart device is in wireless electrical communication (i.e., Wifi, Bluetooth, etc.) with the receiver 132.

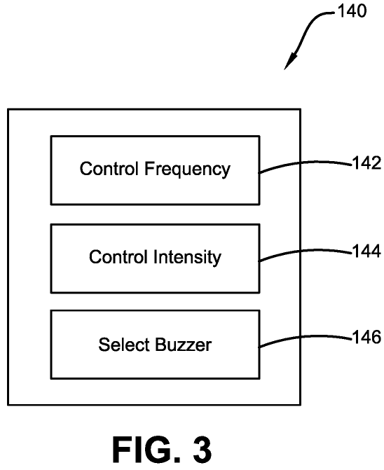

The mobile application 140 can be used by a therapist to remotely control the vibration of the motor 124. More specifically, the application 140 allows a user to control the frequency of vibration 142 of the motor 124, as seen in FIG. 3. The application 140 also allows a user to control the intensity of the vibration 144 of the motor 124. In an embodiment wherein the device 100 is comprised of two buzzers 110 that are in wired 130 or wireless communication, the application 140 may allow a user to select which buzzer 10 receives the vibration 146 from the motor 124. As a result, the therapist can remotely and virtually use the application 140 and buzzer 110 to bilaterally stimulate a user during an EMDR therapy session.

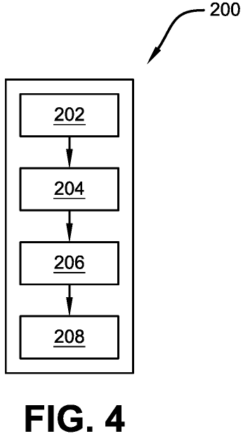

The present invention is also comprised of a method of using 200 the device 100, as seen in FIG. 4. First, a device 100 is provided comprised of at least one buzzer 110 comprised of at least one motor 124 having at least one battery, at least one receiver 132, and a mobile application 140 [Step 202]. Then, the receiver 132 can be paired with the mobile application 140 via wireless electrical communication [Step 204]. Next, a first user can hold the buzzer 110 in their hand [Step 206]. Finally, a second user (such as, but not limited to, a therapist) can use the mobile application 140 to configure the motor 124 to deliver at least one vibration to the first user via the buzzer 110 during a remote, virtual EMDR therapy session [Step 208].

In one embodiment, the device 100 is comprised of two buzzers 110 (wherein one can be held in each hand) and wherein the buzzers 130 are connected via at least one wire 130 and/or wirelessly.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “EMDR therapy tactile stimulation device” and “device” are interchangeable and refer to the EMDR therapy tactile stimulation device 100 of the present invention.

Notwithstanding the foregoing, the EMDR therapy tactile stimulation device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the EMDR therapy tactile stimulation device 100 as shown in the FIGS. are for illustrative purposes only, and that many other sizes and shapes of the EMDR therapy tactile stimulation device 100 are well within the scope of the present disclosure. Although the dimensions of the EMDR therapy tactile stimulation device 100 are important design parameters for user convenience, the EMDR therapy tactile stimulation device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An EMDR therapy tactile stimulation device comprising:
a buzzer comprised of a body comprised of a contoured area;
a motor;
a button;
a battery;
a receiver; and
a mobile application.

2. The EMDR therapy tactile stimulation device of claim 1, wherein the motor is comprised of a vibration motor.

3. The EMDR therapy tactile stimulation device of claim 2, wherein the mobile application controls a frequency of a vibration of the motor.

4. The EMDR therapy tactile stimulation device of claim 2, wherein the mobile application controls an intensity of a vibration of the motor.

5. The EMDR therapy tactile stimulation device of claim 1, wherein the battery is removable from the body.

6. The EMDR therapy tactile stimulation device of claim 1, wherein the battery is rechargeable.

7. An EMDR therapy tactile stimulation device comprising:
a buzzer comprised of a body comprised of a contoured area;
a motor;
a button;
a battery;
an indicator light;
a receiver; and
a mobile application that controls the motor.

8. The EMDR therapy tactile stimulation device of claim 7, wherein the motor is comprised of a vibration motor.

9. The EMDR therapy tactile stimulation device of claim 8, wherein the mobile application controls a frequency of a vibration of the motor.

10. The EMDR therapy tactile stimulation device of claim 8, wherein the mobile application controls an intensity of a vibration of the motor.

11. The EMDR therapy tactile stimulation device of claim 7, wherein the battery is not removable from the body.

12. The EMDR therapy tactile stimulation device of claim 7, wherein the battery is rechargeable via a charging port of the battery.

13. The EMDR therapy tactile stimulation device of claim 7, wherein the indicator light displays a battery level of the battery.

14. The EMDR therapy tactile stimulation device of claim 7, wherein the indicator light displays when the motor is on.

15. A method of using an EMDR therapy tactile stimulation device, the method comprising the following steps:
providing an EMDR therapy tactile stimulation device comprised of a buzzer comprised of a motor having a battery, a receiver, and a mobile application;
pairing the receiver with the mobile application;
having a first user hold the buzzer in a hand; and
having a second user use the mobile application to configure the motor to deliver a vibration during a virtual EMDR therapy session.

16. The method of using an EMDR therapy tactile stimulation device of claim 15, wherein the mobile application controls a frequency of a vibration of the motor.

17. The method of using an EMDR therapy tactile stimulation device of claim 15, wherein the mobile application controls an intensity of a vibration of the motor.

18. The method of using an EMDR therapy tactile stimulation device of claim 15, wherein the mobile application can be installed on a smart device.

19. The method of using an EMDR therapy tactile stimulation device of claim 15, wherein the buzzer is comprised of a contoured area.

20. The method of using an EMDR therapy tactile stimulation device of claim 15, wherein the buzzer is comprised of a button.
